(19) United States
(12) Patent Application Publication
(10) Pub. No.: US 2005/0159670 A1
(43) Pub. Date: Jul. 21, 2005

(54) Method for imaging information, change in information, and computation in the brain

(75) Inventor: Robert Shaw Sneddon, Pasadena, CA (US)

Correspondence Address: Robert S. Sneddon;Apt. 6, 595 North Garfield, Pasadena, CA, 91101, US

(73) Assignee: Sneddon & Associates Inc.

(21) Appl. No.: 11/016,331

(22) Filed: Dec. 18, 2004

Related U.S. Application Data
(60) Provisional application No. 60/530,338, filed on Dec. 18, 2003.

(51) Int. Cl. A61B 5/04

(52) U.S. Cl. 600/544

(57) ABSTRACT

Standard brain imaging methods portray neurological properties such as perfusion (blood flow), sucrose uptake, or basic anatomical features. These depictions are all secondary to the primary function of the brain which is information processing and computation. The present invention provides for an imaging method based directly on brain function. It images the amounts and changes of information in the function of the brain. This is accomplished my measuring brain information at small time and spatial (anatomical) intervals. At each of these intervals, an informational value is computed. The changes in these values are computed by using known neurophysiological constraints, statistical optimization, or a combination of these two methods. These changes can then be depicted in tomographs, if the user so desires. The result is a clear description of brain function at the primary level.

### CROSS-REFERENCE TO RELATED APPLICATIONS

The present patent document claims the benefit of the filing date under 35 U.S.C. §119(e) of Provisional U.S. Patent Application Ser. No. 60/530,338 filed Dec. 18, 2003, which is hereby incorporated in its entirety by reference.

### BACKGROUND OF THE INVENTION

This invention relates to the imaging of biological functions especially neurological processes.

The prior art of imaging does not look at brain information processing, changes, transmutations and computations directly. Yet these are the fundamental processes of the brain, its primary function. Instead, imaging looks at anatomical changes, perfusion (blood flow measured by SPECT), oxygen uptake (fMRI) etc. These processes are related to the brain's function but are only indirect measures. The present invention solves this problem of indirection by imaging a brain's information processing.

In neurological disorders, especially degenerative diseases, there is a standard sequence of neurological losses. The first loss is the ability to process information at normal levels. The second loss is the loss of various types of anatomical function such as decreased perfusion, and decreased sucrose uptake. The final loss is actual cell death and cortical atrophy. Traditional imaging methods do not measure changes in at the beginning of this process. Instead, they only are effective at the second and third stages.

The present invention examines the brain from the point of view of information processing. Attendant to this method is detection and portrayal of brain dysfunction and disorders. This detection occurs at the first step of a degenerative disease, effecting the early detection of this disease. In turn, early detection greatly improves the efficacy of treatment.

### BRIEF SUMMARY OF THE INVENTION

Tomographic methods for examining neurological function are based on either anatomical analysis or correlates of function such as sucrose uptake. They are hindered by an inability to directly measure neurological function related to information processing. The present invention provides a solution to this problem by directly measuring neurological information and its changes as a function of EEG electrode positions or brain anatomy.

The data from an EEG recording is examined in a plurality of time steps and electrode positions. For each time step and each electrode position, an information measure is computed. These values are used to compute changes in information including gain and loss of information; by relating changes in the values over time and electrodes. This has the unexpected result of describing information, flow, loss and gain as a function of time and EEG electrode position or brain anatomy. It has the further result of directly describing brain information processing. These results can be used to describe brain function. They can also be used for the diagnosis, detection and monitoring of neurological conditions including diseases and disorders.

### BRIEF DESCRIPTION OF THE DRAWINGS

FIG. 1 is a Flow Chart for an embodiment of the invention.

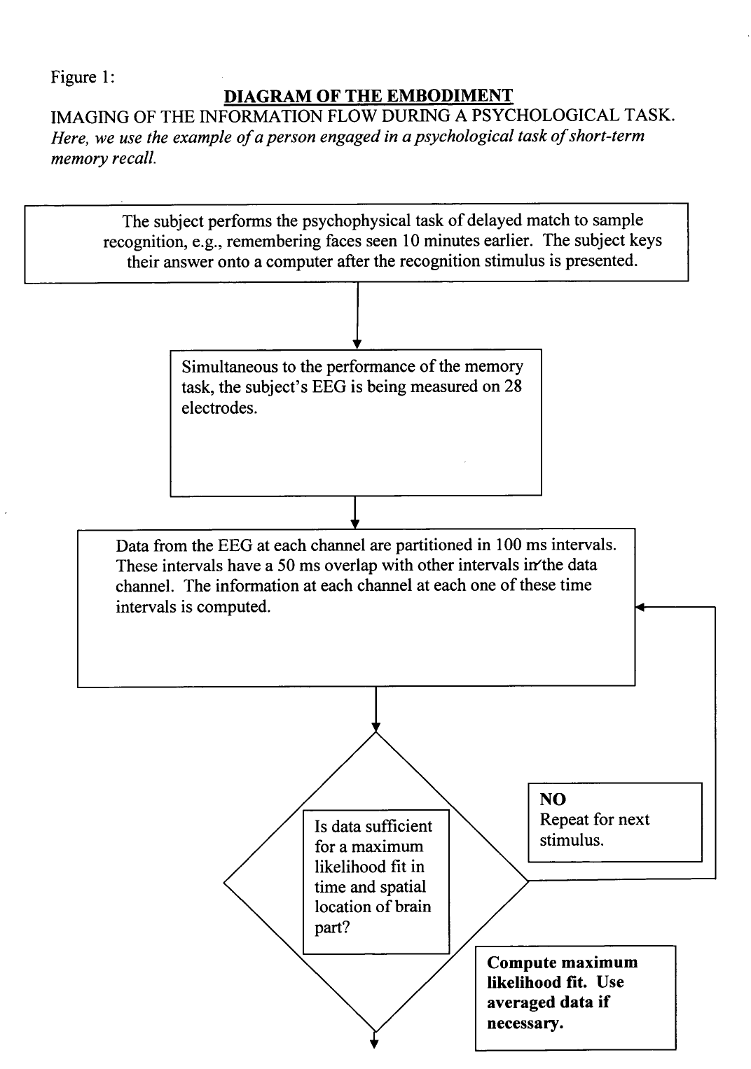

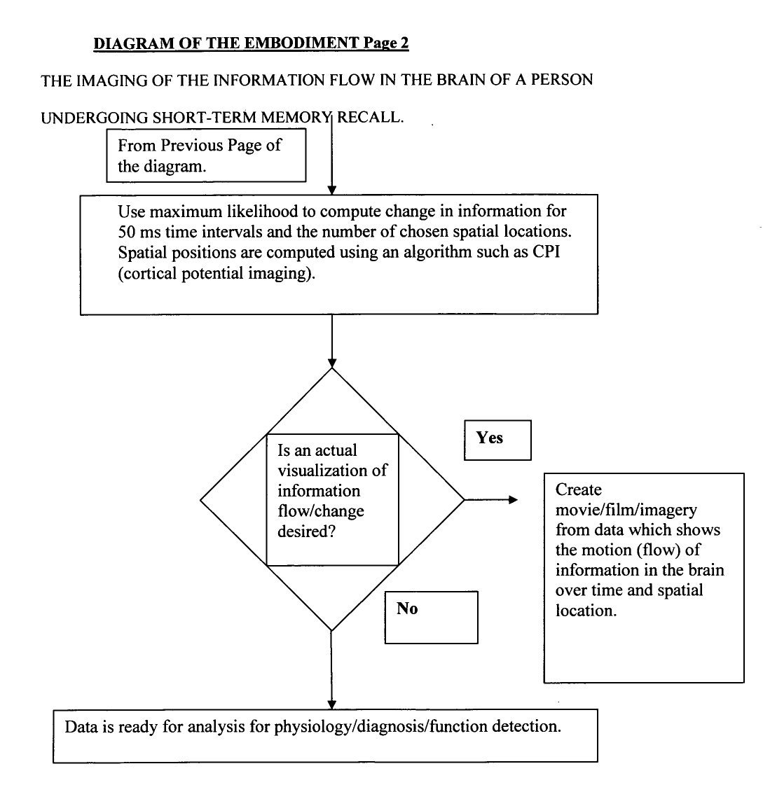

### DETAILED DESCRIPTION OF THE INVENTION AND THE PREFERRED EMBODIMENT

By the present invention, there is provided a method and means of the unexpected ability for imaging biological information especially brain information and its changes over the time course of a task performed by the individual. This provides for enhanced techniques of quantitative EEG, MRI, fMRI, CAT, SPECT etc. The invention provides knowledge which is directly related to the brain's function as an information processor which can be associated with specific diseases and syndromes and thus can assist in establishing different diagnoses.

The determination of the information and its change over time of a brain process consists of first obtaining the ERP data of an individual undergoing a task. The ERP data is examined in specific, short, time intervals in a manner congruent with the neurophysiology of the brain. The information is computed at a first time interval for selected brain regions. It is then compared to the information computed at a second, short time interval for selected brain regions.

The first data and the second data are compared in a manner to compute the change in information at each electrode point or each cortical point beneath the electrodes in a manner which can be purely statistical such as maximum likelihood, or a manner which is congruent with known brain neurophysiology, or a manner which is a combination of statistics and known brain neurophysiology.

The value computed is the change in information value or a value related to information. The quantification of this change allows for the computation of vectors of information change. These vectors can be located on the scalp or within some region of the brain.

The appropriate quantification consists of accounting for three considerations:
1. Information Transfer
2. Information Production
3. Information Loss
   If the representation of the information has been changed, then a fourth consideration is:
4. Information Transmutation

In the preferred embodiment, let the neurological data be data taken from a electroencephalography machine using the standard 10-20 method placing the electrodes as well as placing extra electrodes on the scalp over the areas of the dorso-lateral prefrontal cortex (DLPFC).

We want to examine the change in information in a manner largely congruent with the neurophysiology of working memory. For this reason we examine EEG data taken from a person who is performing a brief working-memory task repeatedly. To examine change in the information as it moves up the dorsal stream, we first examine EEG data taken by the electrodes P7 and P8, because they are place on the scalp area of posterior parietal cortex which forms an early part of the dorsal stream. We would examine this data for about the first 150 milliseconds (ms) after the onset of a working memory stimulus. For each stimulus occurrence and each 150 ms we would compute an informational measure. We might choose to use the method of provisional patent application Ser. No. 60/529,944, “A method to measure information in natural data.”

For the next 150 ms, we would examine the EEG data at the electrodes overlying DLPFC. We would compute the informational measure for these data. Similarly, we may choose to examine information during the feedback phase of this task. That is, examining EEG data recorded from electrodes located on the scalp area above parietal and temporal cortex for the next 150 ms.

Then we would compute changes in information. This would be done by using the P3 and P4 information values as normalization values. The next values, DLPFC left and right hemisphere could be normalized against the P3 and P4 values. And similarly the feedback values would be normalized against the DLPFC values. Statistical accuracy in computing would be reached by using the data from all tasks to compute averages changes over the three time periods, 0-150 ms, 151-300 ms and 301-450 ms.

These changes could be graphically represented on a three-dimensional rendering of the brain. The changes would depict informational activity as moving up the dorsal stream, reaching the DLPFC, where normally this information gets larger, due to ventral inputs. Then the informational activity would be diagrammed as moving back through the proper feedback paths for the next 150 ms.

As a simple example, consider the information of a stimulus moving along the dorsal stream from brain area V1 towards the anterior portion of brain cortex. The neurophysiology of the dorsal stream allows.

In an alternative embodiment, the user obtains data of brain activity or other data which denotes any form of brain measurement including, but not limited to Electroencephalogram, Positron Emission Tomography, Magnetic Resonance Imaging, functional Magnetic Resonance Imaging, Computer Aided Tomography, SPECT etc.

Information or an informational type of measure or approximation thereof is computed from the obtained data using a method such as that described in the provisional patent, “A method for measuring information which has an unknown representation,” inventor: Robert Sneddon. Such information or informational type of measure or approximation thereof is computed at time intervals and spatial locations of the user's choice in accordance with their choice of topography. This choice can be, but is not limited to, brain and/or cranial and/or head points which are most appropriate for that which is to be imaged. This choice can be, but does not have to be, statistically optimal and/or appropriate; or physiologically appropriate or some combination thereof.

These data, computed from the information or informational type of measure or approximation thereof, in accordance with the user's choice of method, is fitted to the user's choice of topography. This choice can, but does not need to be brain topography. This fitting is a fitting for the topographic denotation of information and/or information change and/or computation and/or physiological and/or anatomical topographical portrayal. Said portrayal can but need not be visual. It can, but does not have to be, informational. This fitting is done using knowledge of brain physiology or using a fitting algorithm such as, but not limited to maximum likelihood, or any possible combination of some or all of these techniques. These fitting methods are used to compute information or information-like flow and/or compute information or information-like or an approximation thereof change, and/or measurement of computation and/or physiological and/or anatomical portrayal.

Said changes are localized to specific areas at specific times of the user's chosen topography. The user will achieve localization of said changes by directly using their knowledge of the spatial and temporal properties of said changes and/or by applying a statistical or statistic-like variant algorithm to said changes and/or use knowledge of brain physiology or any possible combination of the aforementioned methods.

Whereas the present invention has been described in particular relation to the drawings attached hereto, it should be understood that other and further modifications apart from those shown or suggested herein, may be made within the scope and spirit of the present invention.

1. A method for describing the changes in informational values by:
a. Separating neurological data into a plurality of intervals based on different times and different portions of the nervous system
b. Computing an informational measure for a plurality of these intervals
c. Computing changes of this informational measure over time and over different potions of the nervous system

2. The method of claim 1 where the changes in the informational measure are computed in the manner of statistical optimization

3. The method of claim 1 where the changes in the informational measure are computed in a manner which is congruent with known neurophysiology

4. The method of claim 1 where the changes in the informational measure are computed in a manner which is congruent with known anatomy

5. The method of claim 1 where the changes in the informational measure are computed in a manner which combines statistical optimization with known neurophysiology

6. The method of claim 1 where said changes are depicted by tomography

7. The method of claim 1 where said neurological data is collected while an individual is performing a neurological or behavioral task

8. The method of claim 1 where said neurological data is electroencephalogram (EEG) data

9. A method for showing the processing of nervous system informational values by:
a. Measuring nervous system activity
b. Estimating information increases, decreases and transmutations from said activity

10. The method of claim 9 where describing said increases, decreases and transmutations are done temporally and spatially

11. The method of claim 10 where temporal and spatial descriptions are related to the neuroanatomy of the brain

12. The method of claim 10 where temporal and spatial descriptions are related to the neurophysiology of the brain

13. The method of claim 10 where temporal and spatial descriptions are computed in a manner which is substantially statistically optimal

14. The method of claim 10 which uses both neurophysiology and statistical analysis

15. The method of claim 10 applied to temporally dynamic data.

16. The method of claim 15 applied to economic data.

17. The method of claim 10 applied to fMRI data.

18. The method of claim 10 with error correction

19. The method of claim 10 applied to data taken from the same source but with different resolutions

20. The method of claim 10 substantially optimized for incomplete data sets
